(19) United States
(12) Patent Application Publication
(10) Pub. No.: US 2010/0325948 A1
(43) Pub. Date: Dec. 30, 2010

(54) SYSTEMS, METHODS, AND MEDIA FOR CIRCULATING AND CARBONATING FLUID IN AN ALGAE CULTIVATION POND

(76) Inventors: Mehran Parsheh, Hayward, CA (US); Jordan Smith, Sacramento, CA (US); Stephen Strutner, San Jose, CA (US); Guido Radaelli, Oakland, CA (US)

Correspondence Address: CARR & FERRELL LLP, 120 CONSTITUTION DRIVE, MENLO PARK, CA, 94025, US

(21) Appl. No.: 12/494,179

(22) Filed: Jun. 29, 2009

(51) Int. Cl. A01H 13/00; A01G 7/00

(52) U.S. Cl. 47/1.4

(57) ABSTRACT

Systems, methods and media for carbonation of fluid in an algae cultivation pond via the use of jets are disclosed. Carbon dioxide is provided to a pressurized fluid. A jet of carbonated fluid is generated from the pressurized fluid and the carbon dioxide. Circulation of the fluid in the algae cultivation pond is initiated via the jet of carbonated fluid.

### FIELD OF INVENTION

The present invention relates generally to the carbonation of fluids, and more particularly to the use of jets for initiating the carbonation of fluid in an aquaculture, such as an algae cultivation pond.

### BRIEF SUMMARY OF THE INVENTION

Provided herein are exemplary systems, methods and media for carbonation of fluid in an algae cultivation pond via the use of jets. In a first aspect, a method for initiating carbonation of fluid in an algae cultivation pond is disclosed. Carbon dioxide is provided to a pressurized fluid. A jet of carbonated fluid is generated from the pressurized fluid and the carbon dioxide. Circulation of the fluid in the algae cultivation pond is initiated via the jet of carbonated fluid.

In a second aspect, a system for initiating carbonation of fluid in an algae cultivation pond is disclosed. The generating fluid flow via a jet in an algae cultivation pond is disclosed. The system includes a series of nozzles submerged in the algae cultivation pond. The series of nozzles is coupled to a source of pressurized fluid and a carbonation source. The series of nozzles is configured to generate a jet of carbonated fluid from the pressurized fluid and the carbonation source and initiate circulation of fluid in the algae cultivation pond via the jet of carbonated fluid. The system includes a pH sensor configured to measure a pH of the fluid in the algae cultivation pond.

In a third aspect, a system for initiating carbonation of fluid in an algae cultivation pond is disclosed. The system includes a series of nozzles submerged in the algae cultivation pond. The series of nozzles is coupled to a source of pressurized fluid and a carbonation source. The series of nozzles is configured to generate a jet of carbonated fluid from the source of pressurized fluid and the carbonation source, and initiate circulation of the fluid in the algae cultivation pond via the jet of carbonated fluid. The system includes a processor and a computer-readable storage medium coupled to the processor, the computer-readable storage medium having embodied thereon a program executable by the processor to perform a method for adjusting a concentration of carbon dioxide in the algae cultivation pond. The processor executes instructions on the computer-readable storage medium to measure a pH associated with the carbonated fluid, and adjust a concentration of carbon dioxide in the jet based on the measured pH.

The methods described herein may be performed via a set of instructions stored on storage media (e.g., computer readable media). The instructions may be retrieved and executed by a processor. Some examples of instructions include software, program code, and firmware. Some examples of storage media comprise memory devices and integrated circuits. The instructions are operational when executed by the processor to direct the processor to operate in accordance with embodiments of the present invention. Those skilled in the art are familiar with instructions, processor(s), and storage media.

### BRIEF DESCRIPTION OF THE DRAWINGS

FIG. 1 illustrates an exemplary jet circulation and carbonation system in accordance with embodiments of the present invention.

FIG. 2 illustrates an embodiment of a jet array distribution system as described in the context of FIG. 1.

FIG. 3 illustrates a method for initiating carbonation of fluid in an algae cultivation pond in accordance with embodiments of the invention

FIG. 4 is a photograph of jet entrainment of a co-flow in an algae cultivation pond in accordance with embodiments of the invention.

### DETAILED DESCRIPTION

Provided herein are exemplary systems, methods and media for providing carbon dioxide to fluid in an algae cultivation pond. The fluid in the algae cultivation pond, i.e. algae cultivation pond fluid, may provide dissolved nutrients and/or raw materials to algae suspended therein. For instance, algae cultivation pond fluid may be composed of a mixture of fresh water and seawater, nutrients to promote algae growth, dissolved gases, disinfectants, waste products, and the like. The uptake of carbon dioxide from fluid in the algae cultivation pond may facilitate photosynthesis, resulting in the accumulation of products such as algal biomass, lipids, and oxygen. The algae cultivation pond may exploit the natural process of photosynthesis in order to produce algae for high-volume applications, such as the production of biofuels.

In order to replenish the carbon dioxide consumed during photosynthesis, carbon dioxide may be introduced in a region of the algae cultivation pond. The carbon dioxide may be introduced to the algae cultivation pond via a jet circulation system that issues jets of carbonated fluid. A jet of carbonated fluid may entrain a co-flow of fluid in the algae cultivation pond, yielding a substantially homogeneous mixture downstream from the jets. The jet entrainment may promote the diffusion and/or advection of carbon dioxide into the algae cultivation pond fluid. The resultant flow associated with one or more jets, i.e. jet flow, may induce bulk movement of fluid in the algae cultivation pond, i.e. circulation, or pond flow.

The use of a jet circulation system in an algae cultivation pond may provide several unexpected advantages that in turn, may raise the productivity, i.e. algal yield per unit area, of the algae cultivation pond. For example, a jet circulation system may accommodate for head losses associated with flow velocities greater than or equal to eight cm/s. The jet circulation system may promote uniform velocity in algae cultivation pond fluid, which may account for lower head losses in the algae cultivation pond. Uniform flow velocity in the algae cultivation pond may promote homogeneity in the algae cultivation pond fluid. Increased homogeneity may promote, for example, enhanced delivery of nutrients, dissolved gases such as carbon dioxide, and/or enhanced temperature distribution in the algae cultivation pond fluid. Uniform flow velocity may also reduce stagnation of fluid in the algae cultivation pond. Reduced stagnation of fluid associated with uniform flow velocity may prevent “dead zones,” or regions of low algal productivity.

The use of a jet circulation system may increase turbulence intensity in the algae cultivation pond fluid. Increases in turbulence intensity may promote the release of byproducts that may be dissolved in the algae cultivation pond fluid. For instance, the increased turbulence intensity may promote the release of dissolved oxygen, which is produced by the algae during photosynthesis. As a result, photosynthetic efficiency of the algae may increase and higher algal yields may be realized. The jets may provide large scale vortices with high energy content to the algae cultivation pond fluid such that the increased turbulence intensity may be sustained far downstream of the nozzle outlet.

Increases in turbulent kinetic energy may promote small-scale fluctuations in the flow velocity of algae cultivation pond fluid, which in turn increase the rate-of-rotation and fluctuating rate-of-strain of the flow. Such fluctuations in rate-of-strain promote the formation of eddies, which encourage vertical and lateral mixing of algae cultivation pond fluid. High turbulence intensity in the flow downstream of the jets enhances the rate of mixing in the pond leading to a more uniform distribution of carbon dioxide across the pond width. Increases in turbulent kinetic energy may result in a turbulent boundary layer at the algal cell and enhance the rate of mass transfer to the algal cells, thereby enhancing the uptake of various nutrients and carbon dioxide.

In some embodiments, the entrainment of algae cultivation pond fluid into the jets may be maximized. Jet entrainment may be significantly increased by generating large scale coherent vortices, in particular, vortex rings. The formation of vortex rings may be induced by the roll-up of the jet shear layer. Increased roll-up of the jet shear layer may occur when the boundary layer in the nozzle from which the jet is issued is laminar. The characteristics of the pond flow into which the jet is issued may affect the jet shear layer and therefore the roll-up of the jet shear layer.

Introducing carbon dioxide to algae cultivation pond fluid, i.e. carbonation, via the use of jets may present additional benefits. The jet entrainment of the pond flow may allow for enhanced delivery and uptake of carbon dioxide by the algae. Enhanced homogeneity due to jet circulation may impede the development of undesirable concentration gradients in the algae cultivation pond fluid. In addition, the use of jets may promote a high level of carbon dioxide dissolution in the algae cultivation pond fluid, thereby reducing or even eliminating carbon dioxide dissipation into the surrounding environment.

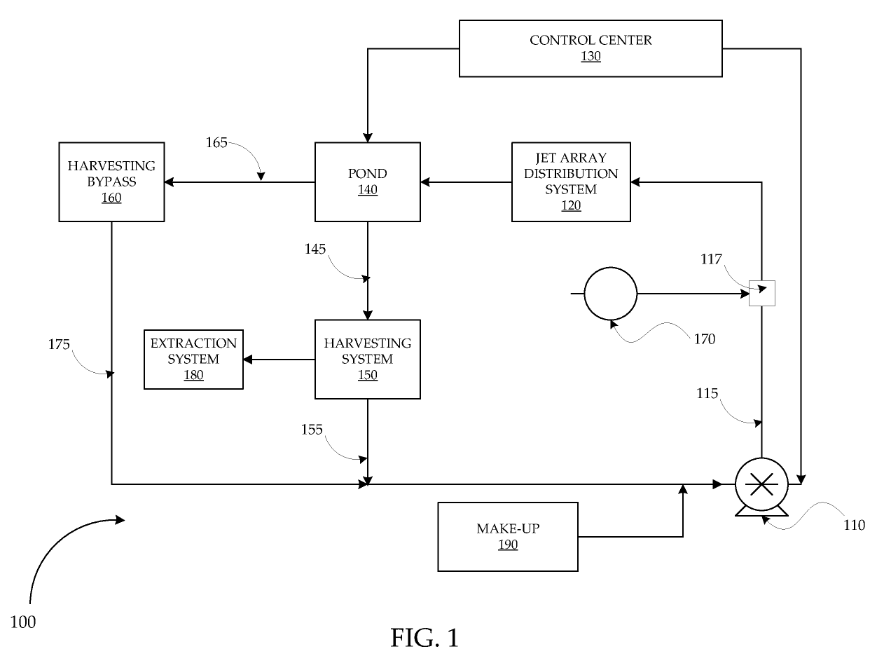

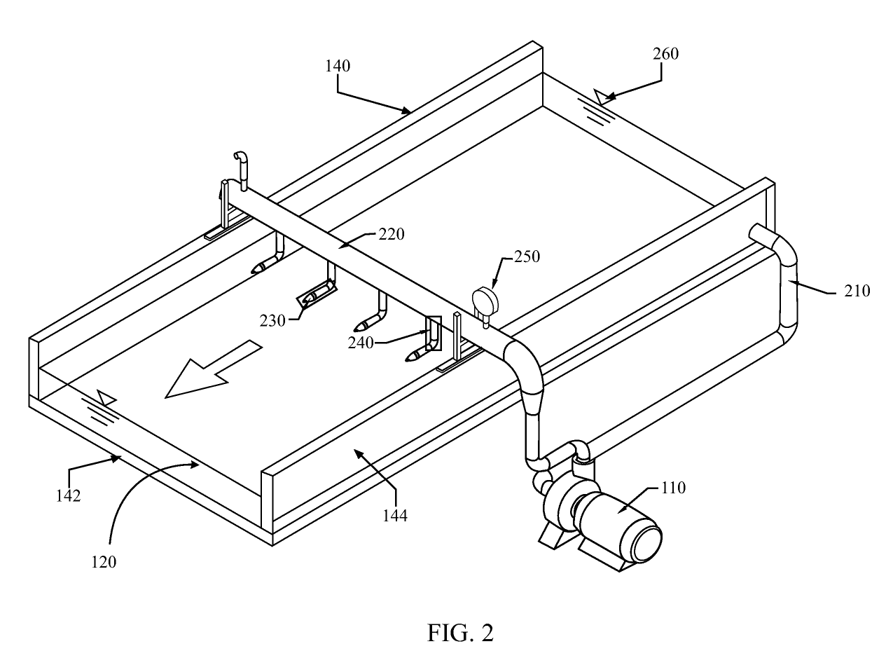

FIG. 1 illustrates an exemplary jet circulation and carbonation system 100. The jet circulation and carbonation system 100 includes a pump 110, a jet array distribution system 120, a control center 130, a pond 140, a harvesting system 150, a harvesting bypass 160, a carbonation source 170, an extraction system 180, and a make-up 190. The pump 110 may be, for example, a centrifugal pump. The jet array distribution system 120 is coupled to the pump 110 and the carbonation source 170 and configured to generate jets of carbonated fluid from the same. Further components of the jet array distribution system 120 are illustrated and described in the context of FIG. 2. One skilled in the art will appreciate that any number of items 110-190 may be present in the jet circulation system 100. For instance, any number of jet array distribution systems 120 may be present in a pond 140, and multiple ponds 140 may be present in jet circulation and carbonation system 100. For all figures mentioned herein, like numbered elements refer to like elements throughout.

In some embodiments, fluid may be pumped from the pump 110 to the jet array distribution system 120 via a path 115. The pump 110 provides energy to move the fluid to jet array distribution system 120, thereby pressurizing the fluid. Carbon dioxide is provided to the jet circulation and carbonation system 100 from the carbonation source 170. The carbonation source 170 may be, for instance, a power plant, a steel mill, a concrete mill, a byproduct of a chemical reaction, and/or any combination of these. The carbonation source 170 may provide pure carbon dioxide in gaseous form or a mixture of gases including carbon dioxide. A portion of the path 115, indicated as an interface 117 in FIG. 1, provides a contact between the pressurized fluid and the carbonation source 170. Upon introduction of the carbon dioxide to the pressurized fluid, the carbon dioxide may dissolve into solution as the pressurized fluid is transported through the path 115. One skilled in the art will recognize that the path 115 may be any length and that the energy provided by the pump 110 may be adjusted in order to optimally dissolve the carbon dioxide in the pressurized fluid.

The jet array distribution system 120 may generate jets of carbonated fluid from the pressurized fluid and the carbon dioxide and discharge the jets into the pond 140. The flow associated with the discharged jets, or jet flow, may have a higher dynamic pressure due to the increased energy generated by the pump 110. The fluid from the jets may entrain the algae cultivation pond fluid (not shown in FIG. 1) and produce a homogeneous mixture of algae cultivation pond fluid downstream of the jets. The jet flow, when brought in contact with the algae cultivation pond fluid, which has lower dynamic pressure, may promote circulation of the algae cultivation pond fluid.

The jet circulation and carbonation system 100 may serve as a cultivation system for large quantities of algae. For instance, the jet circulation and carbonation system 100 may be used to cultivate algae for large volume applications, such as in the production of biofuels. The jet circulation and carbonation system 100 as such may be coupled to, for example, a harvesting system 150 and/or an extraction system 180. Algae may be harvested periodically from the pond 140, i.e. an algae cultivation pond. When harvesting is taking place, algae cultivation pond fluid may be routed from the pond 140 via a path 145. Upon harvesting, algae biomass may be routed to an extraction system 180 and algae cultivation pond fluid may be routed to the pump 110 via a path 155. Alternatively, the algae cultivation pond fluid may be discarded (not shown in FIG. 1).

In order to maintain a desired level of algae cultivation pond fluid, a harvesting bypass 160 may be available in jet circulation and carbonation system 100. The harvesting bypass 160 may include an overflow component, which may act as a reservoir for surplus algae cultivation pond fluid (overflow component not shown in FIG. 1). The harvesting bypass 160 may be used to store excess algae cultivation pond fluid when harvesting is not taking place, such as during maintenance and repair, cleaning, or unfavorable weather conditions. In such scenarios, algae cultivation pond fluid may be routed via a path 165 to the harvesting bypass 160, and then via a path 175 to the pump 110.

Components may be added to jet circulation system 110 based on conditions that may play a role in algae cultivation and/or the needs of the particular genus or species of algae being cultivated. For instance, algae cultivation ponds having several acres of exposed surface area may lose large quantities of water via evaporation to the surrounding environment. Evaporation therefore may change concentrations of various nutrients and/or disinfectants in the algae cultivation pond fluid as well as the temperature of the remaining fluid. In order to maintain desired concentrations of these nutrients and/or disinfectants, a make-up 190 may be available in jet circulation system 100. The make-up 190 may introduce additional fresh water, seawater, disinfectants, and/or nutrients such as Aqua Ammonia, Phosphorous solutions, and trace metals, such as Co, Zn, Cu, Mn, Fe and Mo in appropriate concentrations. In some embodiments, the make-up 190 may draw fluid from the harvesting bypass 160 (path not shown in FIG. 1).

The pump 110, the jet array distribution system 120, the pond 140, the harvesting system 150, the harvesting bypass 160, the extraction 180, and the make-up 190 may be controlled and/or otherwise monitored by the control center 130. The control center 130 may include any number of components, i.e. sensors, gauges, probes, control valves, servers, databases, clients, and any combination of these (not shown in FIG. 1 for simplicity). The sensors, servers, databases, clients and so forth may be communicative with one another via any number or type of networks, for example, LAN, WAN, Internet, mobile, and any other communication network that allows access to data, as well as any combination of these. Clients may include, for example, a desktop computer, a laptop computer, personal digital assistant, and/or any computing device. The control center 130 may monitor and/or measure various parameters in the pond 140, such as pH, head velocity, the head loss associated with the pond flow velocity, temperature, nutrient concentration, concentration of disinfectant, algal density, dissolved oxygen content, turbidity, and the like. The control center 130 may display and/or generate reports based on the various parameters measured in the pond 140.

The control center 130 may store and/or execute software programs and/or instructions in order to take action based on the measured parameters. For instance, the control center 130 may execute a module which compares measured parameters from the pond 140 to a desired set of parameters. If the measured parameters are not within a predetermined range of the desired set of parameters (e.g. within ten percent), the control center 130 may make adjustments via execution of a set of instructions (e.g. a software routine), to any of the pump 110, the jet array distribution system 120, the pond 140, the harvesting system 150, the harvesting bypass 160, the extraction 180, and the make-up 190 in order to bring the measured parameters within the predetermined ranges. For instance, if the pH of the algae cultivation pond fluid drops to an undesirable level, e.g. a pH of 4, the control center 130 may provide instructions to bypass the carbonation source 170.

FIG. 2 illustrates an embodiment of jet array distribution system 120 as described in the context of FIG. 1. As shown in FIG. 2, portions of the jet array distribution system 120 may be situated in the pond 140. Components of jet array distribution system 120 may include an intake 210, a manifold 220, a nozzle 230, a downspout 240, and a gauge 250. FIG. 2 further illustrates algae cultivation pond fluid in the pond 140, a surface of which is indicated by a surface level marker 260. The nozzle 230 is submerged in the algae cultivation pond fluid. One skilled in the art will recognize that any number of components 210-260 may be present in jet array distribution system 120.

In some embodiments, algae cultivation pond fluid may be provided to the pump 110 via an intake 210 as shown in FIG. 2. The intake 210 may provide fluid in the algae cultivation pond to the pump 110, as shown in FIG. 2. Alternatively, the intake 210 may provide algae cultivation pond fluid from a component shown in FIG. 1, such as the harvesting system 150, the harvesting bypass 160, and/or the make-up 190. The intake 210 may be coupled to the control center 130, discussed in the context of FIG. 1.

Upon intake of algae cultivation pond fluid, the pump 110 may provide the algae cultivation pond fluid to the manifold 220. The pump 110 provides energy to the algae cultivation pond fluid in order to transport the algae cultivation pond fluid to the manifold. Energy provided by the pump 110 pressurizes the algae cultivation pond fluid. The manifold 220 may distribute the pressurized algae cultivation pond fluid to the nozzles 230. One skilled in the art will recognize that the manifold 220 may be configured to provide algae cultivation pond fluid to any number of nozzles 230 and not just to four nozzles 230 as shown in FIG. 2. For instance, a single nozzle 230 may provide circulation in the algae cultivation pond.

The nozzles 230 may generate jets from the pressurized algae cultivation pond fluid (jets not shown in FIG. 2). A flow associated with the jets may provide kinetic energy to a pond flow in the algae cultivation pond. Per the “Law of Continuity,” the flow in the pond, which includes the jet flow and the entrained co-flow, obtains a velocity from the jet flow. The kinetic energy of the pond flow translates into a higher static pressure. Since the pond flow has a free surface, as indicated by surface level marker 260, the higher static pressure translates into a head, thereby initiating and/or maintaining circulation of algae cultivation pond fluid in the algae cultivation pond.

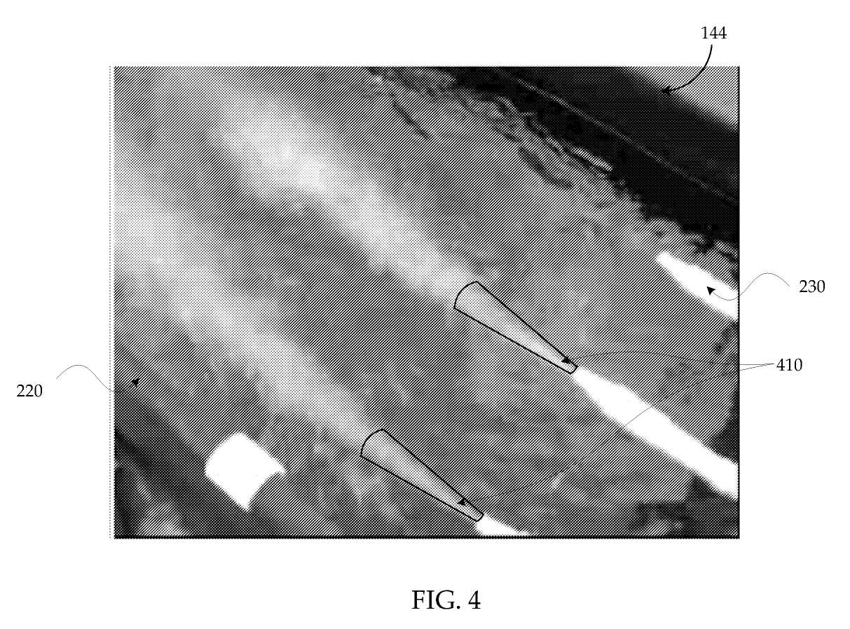

The flow associated with the jets, i.e. jet flow, may entrain the co-flow into the jets downstream of the nozzles 230. The entrainment of the co-flow into the jet flow may allow for distribution of nutrients, dissolved gases, minerals, and the like. In some embodiments, one jet may be issued per nozzle 230, however, multiple jets may issue from a single nozzle 230. An array of jets may be generated from the jet array distribution system 120 based on a placement of nozzles relative to each other. An exemplary nozzle array is further shown in FIG. 4. In some embodiments, the manifold 220 may provide the pressurized algae cultivation pond fluid to the nozzles 230 via optional spouts 240. The spouts 240 may be useful when the manifold is placed above the pond 140 and the nozzles 230 are submerged in the algae cultivation pond fluid as shown in FIG. 2. A plurality of configurations of the manifold 220 beyond those shown in FIG. 2 may be implemented. For instance, the manifold 220 and the nozzles 230 may be submerged in the algae cultivation pond 140. In such embodiments, the manifold 220 may be placed parallel to the configuration shown in FIG. 2, but along the floor 142 of the algae cultivation pond (placement not shown in FIG. 2). Alternatively, the manifold 220 may be placed along a wall 144 of the algae cultivation pond (placement not shown in FIG. 2).

Any number and/or type of gauges and/or sensors 250 may be used to measure various parameters in the jet array distribution system 120. For instance, pH sensors may be coupled to the manifold 220 and measure pH in the manifold (as shown in FIG. 2) and in the pond 140 (not shown in FIG. 2). The pH measurements may be indicative of carbon dioxide concentrations in the algae cultivation pond fluid. The gauges 250 may be coupled to the control center 130, which may store and/or display data associated with the gauges 250. The gauges 250 may be coupled to the control center 130, which may execute algorithms to determine parameters such as flow rate, head loss, temperature, pH, concentration of dissolved gases, turbidity, turbulence characteristics, and the like.

The control center 130 may execute programs and/or software in order to take action on any of components 210-260 based on measured and/or derived data from the gauges 250. For instance, if a pH measurement indicates that the pH in the algae cultivation pond fluid is higher than an acceptable range, the control center 130 may, via execution of an algorithm, take measures to lower the pH of the algae cultivation pond fluid. The algorithm may call for increased carbon dioxide availability from the carbonation source 170. In addition, the algorithm may determine from which of the pond 140, the harvesting system 150, the bypass 160, and/or the make-up 190 to source. One skilled in the art will recognize that a plurality of instructions may be stored and/or executed by control center 130 in order to adjust and/or maintain conditions in the pond 140.

The jet array distribution system 120 may be used in conjunction with an algae cultivation pond of any design. The algae cultivation pond may include any body of water for the purpose of cultivating algae. For instance, the jet array distribution system 120 may be applied to open-air raceway ponds used in the cultivation of Dunaliella or Spirulina, flumes and/or algae channels.

The jet circulation and carbonation system 100 may be customized based on the design of the algae cultivation pond and/or the needs of the particular genus or species of algae being cultivated therein. For instance, each pond 140 may be characterized by a frictional head loss associated with a particular flow velocity. In order to promote circulation in the pond 140, the pump 110 may provide energy, or head, to the jets. As such, the nozzles 230 may be organized in an array such that the resulting jet flow overcomes the frictional head loss associated with the pond 140.

In addition, a plurality of configurations of the nozzles 230 beyond those shown in FIG. 2 may be implemented. These objectives may include maximizing efficiency, maximizing generated head, minimizing the distance over which the pond flow downstream of the nozzles 230 reaches uniform velocity, maximizing turbulence of the fluid flow in the algae cultivation pond, minimizing the effects of “dead zones,” generating energetic vortices of particular frequencies, and any combination of these. An exemplary linear nozzle array is shown in FIG. 2, with the four nozzles in approximately the same depth in the pond 140.

For instance, the nozzles 230 may be placed at any flow depth in the pond 140. Flow depth may be characterized as a perpendicular distance between a surface of the algae cultivation pond fluid, as indicated by surface level marker 260, and the floor 142. Nozzle depth may be characterized as a perpendicular distance between a surface of the algae cultivation pond fluid, as indicated by surface level marker 260, and a nozzle 230. With respect to the embodiments discussed according to FIG. 2, the nozzles 230 are shown in a horizontal array at a substantially uniform nozzle depth. An exemplary depth for the nozzles 230 in the jet array distribution system 120 may be between twenty and thirty centimeters from the surface of the algae cultivation pond fluid, depending on the particularities of the design of the pond 140. A nozzle depth may be characterized relative to the flow depth, i.e. the nozzle depth may be halfway between the surface of the algae cultivation pond fluid (indicated by surface level marker 260) and the floor 142. In such scenarios, the nozzle depth may be characterized as in, or approximately in, the “middle” of the flow depth. Nozzle depth may play a role in the formation of large vortex rings and promote the entrainment of the co-flow into the jet flow. The generation of large scale, high energy vortices may promote the dissolution of carbon dioxide in the algae cultivation pond fluid.

The nozzles 230 may include nozzles of any design that may be configured to issue a submerged jet. In some embodiments, the nozzles 230 may be selected based on flow characteristics. For instance, a laminar boundary layer between fluid in the nozzles 230 and interior surfaces of the nozzles 230 (boundary layer not shown in FIG. 2) from which a jet is issued may promote the formation of vortex rings in the algae cultivation pond fluid. Since the formation of vortex rings in the algae cultivation pond fluid may facilitate entrainment of the co-flow of the algae cultivation pond fluid into the jet flow, ranges of jet flow velocities may be maintained such that a laminar boundary layer is maintained in the nozzles 230. With respect to the embodiments discussed in FIGS. 1 and 2, the ranges of flow velocities may be empirically determined and programmable into a set of instructions that are executable by the control center 130.

The nozzles 230 may be immobile and therefore form a static array. Alternatively, the array may be dynamic. For example, the nozzles 230 may be mobile and therefore various configurations of arrays may be arranged in real-time based on a desired resultant jet flow. In addition, the manifold 220 may be configured to provide pressurized algae cultivation pond fluid to all of the nozzles 230, or to selected nozzles 230 based on a desired jet and/or resultant jet flow. The arrangement of arrays may be managed at the control center 130. The control center 130 may execute instructions to manipulate and arrange various arrays based on a set of criteria, which may include, for example, a desired resultant jet flow, a desired ratio between a resultant jet flow and a background flow (co-flow) in the algae cultivation pond, and the like.

The number of jets forming the jet array may be affected by the design of the particular algae cultivation pond. For instance, the number of jets may be determined based on one of a flow depth of the algae cultivation pond, a desired distance between two jets, a jet diameter (based on characteristics of a cross section of a nozzle from which the jet is issued), a co-flow velocity in the algae cultivation pond, and any combination thereof. For instance, a distance of thirty centimeters between the nozzles 230 may be desired in order to maximize jet entrainment.

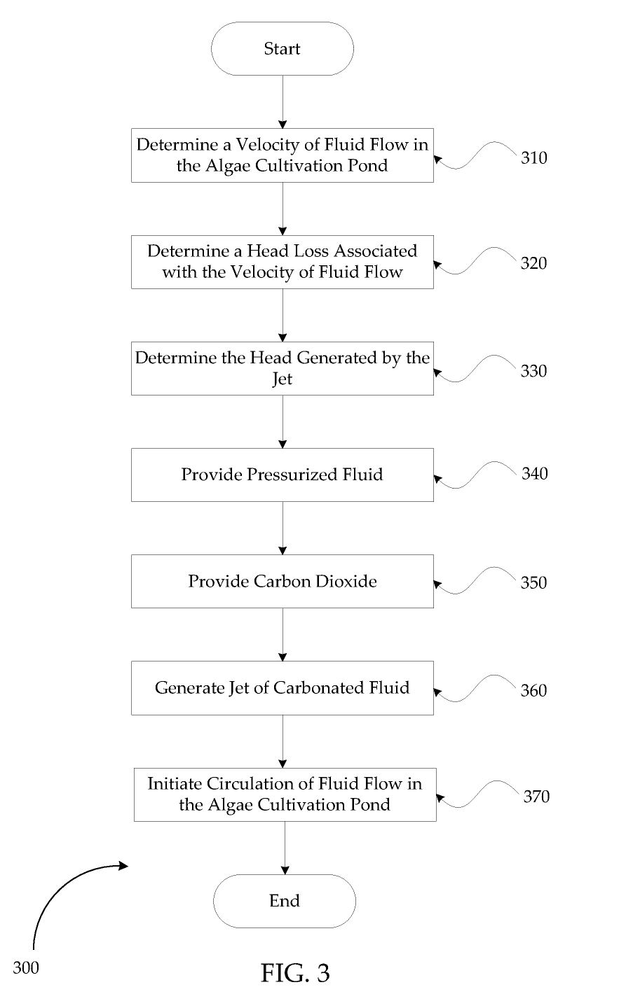

FIG. 3 illustrates a method 300 for initiating carbonation in an algae cultivation pond. In some embodiments, the method 300 may be used to initiate carbonation of algae cultivation pond fluid in the pond 140 via the nozzles 230 and the control center 130, as discussed in the context of FIGS. 1 and 2. In step 310, a velocity for fluid flow in the algae cultivation pond is determined. The velocity for fluid flow in the algae cultivation pond may range from, for example, 8 cm/s to 100 cm/s. In order to reduce the effects of “dead zones” resulting from the jet flow, co-flow velocities of 40 cm/s to 70 cm/s in the proximity of the nozzle outlets may be effective.

In step 320, a head loss associated with the velocity of fluid flow in the algae cultivation pond determined in step 310. The head loss associated with the velocity of fluid flow may be determined based on the particularities of the algae cultivation pond. For instance, the particularities of the pond design and the determined velocity for fluid flow in step 310 may be taken into account. As mentioned earlier, the pond 140 as disclosed in FIGS. 1 and 2 may be an exemplary algae cultivation pond in which the method 300 may be practiced. As such, the head loss of the algae cultivation pond may be characterized as a loss of energy due to friction of fluid along the floor 142, any of the walls 144, as well as along turns and/or bends in the algae cultivation pond which may cause flow separation.

In step 330, the head generated by the jet is determined. The head generated by the jet may be selected so as to overcome the head loss determined in step 320 associated with the velocity for fluid flow determined in step 310. As shown in FIG. 2, the pump 110 may provide energy to the algae cultivation pond fluid in order to transport the algae cultivation pond fluid through the path 115.

In step 340, pressurized fluid is provided. The fluid may be drawn from any reservoir of fluid as illustrated and described in the context of FIG. 1. In step 350, carbon dioxide is provided to the pressurized fluid from step 340. The carbon dioxide, which may be pressurized, may be sourced from the carbonation source 170. The carbon dioxide dissolves in the pressurized fluid and the resulting carbonated fluid is provided to the jet array distribution system 120. Exemplary carbon dioxide pressure ranges for the embodiments disclosed herein range from four to twenty-five pounds per square inch.

The pressures of fluid from step 340 and the carbon dioxide from step 350 may be controlled and balanced by an algorithm executed by the control center 130. For instance, carbon dioxide pressures may be adjusted in step 350 based on fluid pressures in step 340 in order to achieve maximum uptake and dissolution of carbon dioxide by the fluid. The pressures from steps 340 and 350 may be subject to constraints imposed by the algorithm executed by the control center 130. For instance, the concentration of carbon dioxide in the algae cultivation pond fluid may be less than a concentration associated with a saturation point of carbon dioxide in the algae cultivation pond fluid.

In step 360, a jet of carbonated fluid is generated from the pressurized fluid and the carbon dioxide. The concentration of dissolved carbon dioxide in the jet may be monitored and/or otherwise adjusted at the control center 130. For instance, a pH meter may provide a measurement corresponding to carbon dioxide concentration in the algae cultivation pond fluid. Once a desired concentration of carbon dioxide is reached, the injection of carbon dioxide may be terminated. Likewise, if the concentration of carbon dioxide is not within a predetermined range of the desired concentration, the injection of carbon dioxide may be initiated. The concentration of carbon dioxide in the jets, and therefore, in the jet flow, may be higher or lower than the concentration of fluid in the algae cultivation pond depending on the measured carbon dioxide concentration in the algae cultivation pond fluid downstream of the jets.

In step 370, circulation of fluid flow in the algae cultivation pond may be initiated via the jet of carbonated fluid. The submerged nozzles 230 may generate submerged jets of carbonated fluid from the carbonated fluid in steps 340-350. The jets may be discharged into the pond 140, upon which the background flow, or co-flow of the algae cultivation pond, may be entrained into the jets. The entrainment of the jets may promote the distribution of carbonated fluid in the algae cultivation pond fluid, resulting in a homogeneous mixture downstream of the jets. In addition, the jets of carbonated fluid from step 360 may provide kinetic energy to the algae cultivation pond fluid, thereby promoting circulation in the pond 140.

FIG. 4 is a photograph of jet entrainment of a co-flow in an algae cultivation pond in accordance with the embodiments discussed in the context of FIGS. 1, 2, and 3 above. FIG. 4 shows a wall 144 of a pond 140 (i.e. algae cultivation pond), a manifold 220, and three nozzles 230. The pond 140 is filled with algae cultivation pond fluid. FIG. 4 indicates that the nozzles 230 are fully submerged in the algae cultivation pond fluid. Jets 410 are issued from the nozzles 230. As is illustrated in FIG. 4, the jets 410 may entrain the co-flow in an algae cultivation pond, as is shown. The entrainment of the co-flow into the jets as shown in FIG. 4 and the circulation in the pond resulting from the jets may correspond to step 350 in the method 300 discussed above.

The above-described functions and/or methods may include instructions that are stored on storage media. The instructions can be retrieved and executed by a processor. Some examples of instructions are software, program code, and firmware. Some examples of storage media are memory devices, tapes, disks, integrated circuits, and servers. The instructions are operational when executed by the processor to direct the processor to operate in accord with the invention. Those skilled in the art are familiar with instructions, processor(s), and storage media. Exemplary storage media in accordance with embodiments of the invention are discussed in the context of, for example, the control center 130 of FIG. 1. In addition, portions of the method 300 may be embodied in code that is executable by a computer associated with the control center 130.

Upon reading this paper, it will become apparent to one skilled in the art that various modifications may be made to the systems, methods, and media disclosed herein without departing from the scope of the disclosure. As such, this disclosure is not to be interpreted in a limiting sense but as a basis for support of the appended claims.

1. A method for initiating carbonation of fluid in an algae cultivation pond, the method comprising:
providing carbon dioxide to a pressurized fluid;
generating a jet of carbonated fluid from the pressurized fluid and the carbon dioxide; and
initiating circulation of the fluid in the algae cultivation pond via the jet of carbonated fluid.

2. The method of claim 1, further comprising dissolving the carbon dioxide in the fluid in the algae cultivation pond at less than a saturation point in the fluid in the algae cultivation pond.

3. The method of claim 1, wherein the jet of carbonated fluid circulates the fluid in the algae cultivation pond at a velocity of twenty centimeters per second.

4. The method of claim 1, further comprising:
measuring a pH of the carbonated fluid; and
adjusting a concentration of carbon dioxide in the jet based on the measured pH.

5. The method of claim 4, wherein measuring the pH includes measuring a pH of a flow of the algae cultivation pond.

6. The method of claim 1, further comprising generating a velocity of fluid flow of at least eight centimeters per second in the algae cultivation pond.

7. The method of claim 6, further comprising providing a head to the jet that overcomes a head loss associated with the velocity of fluid flow of at least eight centimeters per second in the algae cultivation pond.

8. The method of claim 1, further comprising initiating an entrainment of a flow in the algae cultivation pond into the jet of carbonated fluid.

9. The method of claim 8, wherein initiating an entrainment of a flow in the algae cultivation pond via a plurality of vortices.

10. A system for initiating carbonation of fluid in an algae cultivation pond, the system comprising:
a series of nozzles submerged in the algae cultivation pond, the series of nozzles coupled to a source of pressurized fluid and a carbonation source, the series of nozzles configured to:
generate a jet of carbonated fluid from the pressurized fluid and the carbonation source, and
initiate circulation of fluid in the algae cultivation pond via the jet of carbonated fluid; and
a pH sensor configured to measure a pH of the fluid in the algae cultivation pond.

11. The system of claim 10, the system further comprising a manifold coupled to an inlet of the series of nozzles and to the source of pressurized fluid.

12. The system of claim 11, wherein a pressure in the manifold is between four and twenty-five pounds per square inch.

13. The system of claim 10, wherein the source of carbonation is pure carbon dioxide in gaseous form.

14. A system for initiating carbonation of fluid in an algae cultivation pond, the system comprising:
a series of nozzles submerged in the algae cultivation pond, the series of nozzles coupled to a source of pressurized fluid and a carbonation source, the series of nozzles configured to:
generate a jet of carbonated fluid from the source of pressurized fluid and the carbonation source, and
initiate circulation of the fluid in the algae cultivation pond via the jet of carbonated fluid;
a processor; and
a computer-readable storage medium having embodied thereon a program executable by the processor to perform a method for adjusting a concentration of carbon dioxide in the algae cultivation pond, wherein the computer-readable storage medium is coupled to the processor, the processor executing instructions on the computer-readable storage medium to:
measure a pH associated with the carbonated fluid; and
adjust a concentration of carbon dioxide in the jet based on the measured pH.

15. The system of claim 14, wherein the jet of carbonated fluid circulates the fluid in the algae cultivation pond at a velocity of twenty centimeters per second.

16. The system of claim 14, wherein the method executed by the processor further comprises:
initiating a circulation of fluid in the algae cultivation pond via at least one jet, the circulation of fluid generating a velocity of fluid flow of at least ten centimeters per second in the algae cultivation pond; and
providing a head to the jet that overcomes a head loss associated with the velocity of fluid flow of at least ten centimeters per second in the algae cultivation pond.

17. The system of claim 14, wherein the method executed by the processor further comprises generating a report based on the measured pH.
